(19) United States
(12) Patent Application Publication
(10) Pub. No.: US 2005/0107689 A1
(43) Pub. Date: May 19, 2005

(54) Medical image management system

(75) Inventor: Yasuhiko Sasano, Tokyo (JP)

Correspondence Address: Finnegan, Henderson, Farabow,;Garrett & Dunner, L.L.P., 1300 I Street, N.W., Washington, DC, 20005-3315, US

(73) Assignee: KONICA MINOLTA MEICAL & GRAPHIC, INC.

(21) Appl. No.: 10/984,891

(22) Filed: Nov. 10, 2004

(30) Foreign Application Priority Data
Nov. 14, 2003 (JP) PAT. 2003-385318

(51) Int. Cl. A61B 5/05

(52) U.S. Cl. 600/425

(57) ABSTRACT

A medical image management system includes: an information storage for storing a medical image and report information regarding diagnosis; a displaying section for displaying the medical image and/or the report information from the information storage; an operating section for setting image format of display screen and its displaying turn for displaying the medical image and/or the report information; a pattern storage for storing the display appearance and the displaying turn set through the operating section so as to relate them with user information of an operator of the operating section; an obtaining section for obtaining user information of an interpreting user; and a display controller for obtaining the medical image and/or the report information during the interpretation, and display pattern information corresponding to the user information, and displaying the medical image and/or the report information according to the display appearance and the displaying turn corresponding to the display pattern information.

### BACKGROUND OF THE INVENTION

1. Field of the Invention

The present invention relates to a medical image management system which manages a medical image in which an image of a subject is generated and displays the medical image on a display.

2. Description of Related Art

In a medical field nowadays, there are a lot of situations where a medical image in which an image of a subject is generated is displayed on a display such as an LCD (Liquid Crystal Display) for interpretation. As a medical image, various types of images such as an X-ray image, a CT (Computed Tomography) image which is generated by a CT apparatus, an MRI (Magnetic Resonance Imaging) image which is generated by an MRI apparatus and the like exist. According to digitization of a medical image, it is nowadays possible to display medical images according to various types of display appearances such as: a display appearance in which an X-ray image and a CT image are simultaneously displayed on one screen, a display appearance in which an X-ray image generated in the previous and a current X-ray image are simultaneously displayed, etc.

Even if the same medical image is to be interpreted, a display appearance at the time of the interpretation differs depending upon a region to be diagnosed and/or a personal preference of a doctor. Further, a display appearance also differs depending upon an operation of a doctor such as a doctor who performs the interpretation (hereafter, it is called an interpretation doctor), a doctor who confirms and approves a report created by the interpretation doctor (hereafter, it is called an approval doctor), and the like. However, since it is complicated for a user such as a doctor or the like to set a display appearance according to each interpretation, what has been disclosed recently is a technology which outputs a medical image according to a display appearance being appropriate to a image generating condition such as a region to be radiographed (image-generated) and the like (for example, JP-Tokukai-2002-158862A)

However, the above-mentioned conventional technology only customizes a display appearance on one screen. Therefore, if it is necessary to interpret a plurality of medical images continuously through a plurality of display screens, a display appearance which is appropriate to each screen has to be called up at each time, whereby the operation remains complicated.

### SUMMARY OF THE INVENTION

An object of the present invention is to make it possible to customize a procedure of display operations at the time of displaying a medical image and the like for interpretation, in order to promote the efficiency of interpretation operations.

In accordance with a first aspect of the present invention, a medical image management system comprises: an information storing section for storing a medical image in which an image of a subject is generated and report information regarding diagnosis; a displaying section for displaying at least one of the medical image and the report information each of which is obtained from the information storing section; an operating section for setting an image format of a display screen and displaying turn of the display screen which are used to display at least one of the medical image and the report information on the displaying section; a pattern storing section for storing the display appearance of the display screen and the displaying turn of the display screen which are set through the operating section so as to relate the display appearance and the displaying turn with user information regarding a first user who has operated the operating section; an obtaining section for obtaining user information of a second user who performs an interpretation; and a display controlling section for obtaining at least one of the medical image and the report information from the information storing section at the interpretation, for obtaining display pattern information corresponding to the user information obtained by the obtaining section from the pattern storing section, and for displaying at least one of the medical image and the report information on the displaying section according to the display appearance and the displaying turn corresponding to the obtained display pattern information.

According to the system of the first aspect, display appearances and their displaying turn at the time of displaying at least one of a medical image and report information are set to each user as a display pattern, and at least one of a medical image and report information are displayed according to the display pattern corresponding to a user during an interpretation. Therefore, it is possible to customize display appearances and displaying turn according to a diagnosis purpose of each user and personal preference. Consequently, it is possible to customize a displaying operation during an interpretation, whereby it is possible to improve efficiency of the interpretation operation.

Preferably, the system further comprises a pattern controlling section for collecting statistics of the display pattern information stored in the display pattern storing section, for calculating a display pattern having highest frequency of use as a standard pattern, and for storing information of the standard pattern in the display pattern storing section.

According to the above-mentioned system, display pattern information which is set to each user is stored so as to build a database, and the statistics of the display pattern is collected for calculating a standard pattern having the highest frequency of use to be stored in the database. Therefore, it is possible to standardize a display pattern according to a diagnosis department unit, a hospital unit and the like, whereby it is possible to unify an operation procedure such as interpretation, approval and the like. Further, by performing the interpretation operation based on such a standard pattern, it is possible to efficiently perform the interpretation even for an interpretation doctor having little experience.

### BRIEF DESCRIPTION OF THE DRAWINGS

The present invention will become more fully understood from the detailed description given hereinafter and the accompanying drawing given by way of illustration only, and thus are not intended as a definition of the limits of the present invention, and wherein:

FIG. 1 is a view showing a system structure of a medical image management system 100 according to the present embodiment,

FIG. 2 is a view showing an internal structure of a server 20,

FIG. 3 is a view showing a data structure example of a display pattern DB 29,

FIG. 4 is a view showing an internal structure of an interpretation terminal 40,

FIG. 5 is a flowchart illustrating a tracing process performed by the interpretation terminal 40,

FIGS. 6A to 6C are views showing display appearance examples of a list screen,

FIGS. 7A to 7D are views showing display appearance examples of an interpretation screen,

FIGS. 8A to 8C are views showing display appearance examples of a report screen, and

FIG. 9 is a flowchart illustrating a pattern display process performed by the server 10 and the interpretation terminal 40.

### PREFERRED EMBODIMENTS OF THE INVENTION

In the present embodiment, what will be described is an example in which a display appearance of a display screen which is displayed at the time of interpretation on an interpretation terminal and the displaying turn of the display appearance are set as a display pattern according to each user, and the display pattern set in each interpretation terminal is stored as database in a server to be managed.

First, a structure of the present embodiment will be described.

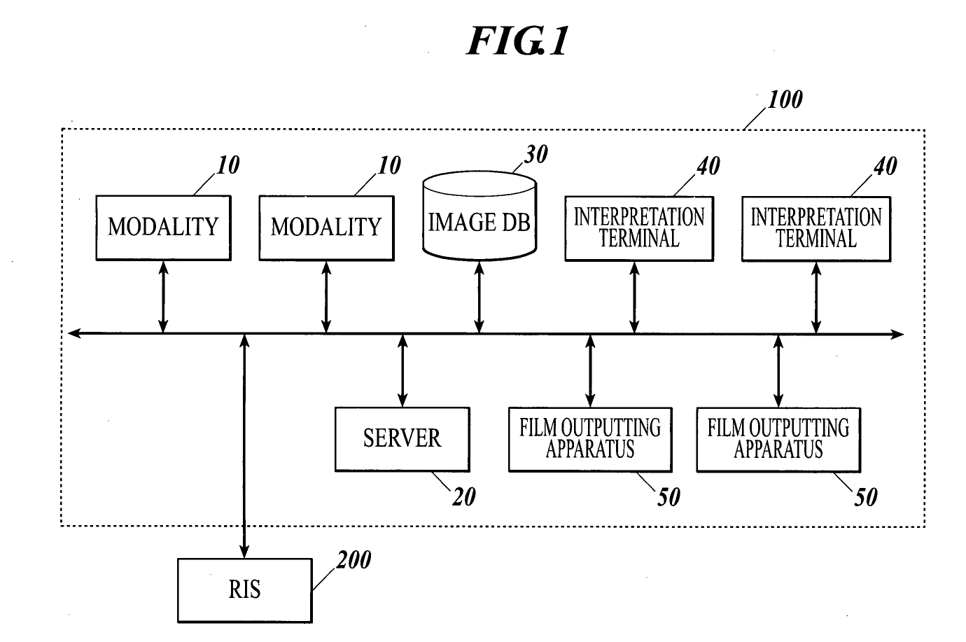

FIG. 1 shows a system structure of a medical image management system 100 according to the present embodiment.

As shown in FIG. 1, the medical image management system 100 comprises a modality 10, a server 20, an image DB (DataBase) 30, an interpretation terminal 40 and a film outputting apparatus 50, where each apparatus is connected through a communication network N so as to make a mutual data transmission/reception possible. Here, in FIG. 1, shown is the case that two modalities 10, two interpretation terminals 40, two film outputting apparatuses 50, one server 20 and one image DB are connected. However, the number of each apparatus is not specifically limited.

Further, the medical image management system 100 is connected to an RIS (Radiography Information System) 200 through the communication network N.

The RIS 200 is an information management system which manages information in a department of radiology. Concretely, in response to a request from a doctor, the RIS 200 generates patient information regarding a patient such as a patient ID, a name, a sex and the like of the patient to be radiographed (image-generated), and radiographing order information including examination information and the like, the examination information such as an examination ID for examination, a radiographic region (region to be image-generated) assigned by the examination, a radiographing (image-generating) method and the like, and adds identification information (hereafter, it is called an order ID) to the generated radiographing order information for management. The RIS 200 transmits the generated radiographing order information to each modality 10 of the medical image management system 100.

Here, in the present embodiment, described is the case that the RIS is applied as an information management system. However, as an information management system, it is possible to apply a reception terminal which accepts an instruction to issue radiographing order information, an HIS (Hospital Information System) which manages information in a hospital, and the like.

Hereinafter, each component of the medical image management system 100 will be described.

The modality 10 is used to generate image data of a medical image in which an image of a subject is generated. For example, a CR apparatus, a film digitizer which reads an X-ray image recorded in an X-ray film, and a reading apparatus which reads an X-ray image from a cassette in which the X-ray image is recorded, can be applied as the modality 10. In addition, a modality which generates various image types of medical images, such as an MRI apparatus, a CT apparatus, an apparatus for generating an ultrasonogram and the like can be applied as the modality 10.

The modality 10 radiographs (generates an image of) a subject and generates image data of the medical image according to radiographing order information generated by the RIS 200. Then, as accompanying information, the modality 10 adds patient information and examination information for identifying the medical image, to the generated image data of the medical image. Further, the modality 10 adds series information regarding a name of the modality 10, an operator name, an examining part and the like, image information such as date and time of generating the medical image, bit information and the like, to the image data. Then, the modality 10 transmits the image data to which the accompanying information, the series information and the image information are added, to the server 20.

The server 20 stores the image data of the medical image transmitted from the modality 10, in the image DB 30 as an original image. Further, the server 20 comprises a large-capacity storing section. In the storing section, the patient information and the examination information which are previously obtained from the RIS 200 are rewritably stored as fundamental information so as to build a database. Further, the series information and the image information included in the accompanying information of the medical image which is newly inputted is stored as fundamental information regarding a medical image so as to build a database.

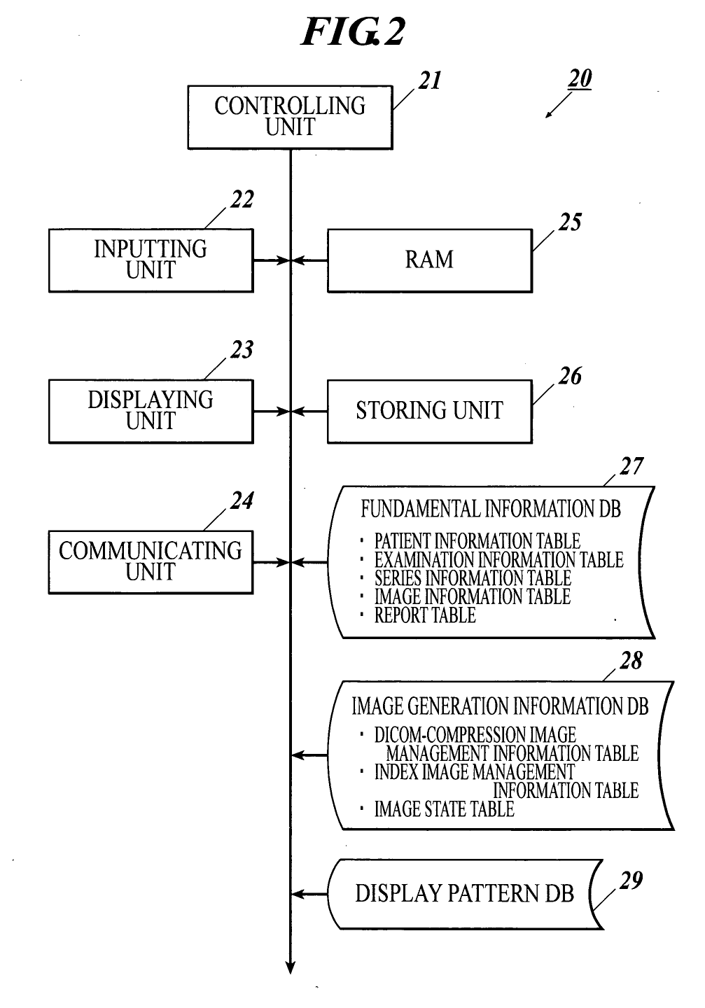

FIG. 2 shows an internal structure of the server 20.

As shown in FIG. 2, the server 20 comprises a controlling unit 21, an inputting unit 22, a displaying unit 23, a communicating unit 24, a RAM (Random Access Memory) 25, a storing unit 26, a fundamental information DB 27, an image generation information DB 28 and a display pattern DB 29.

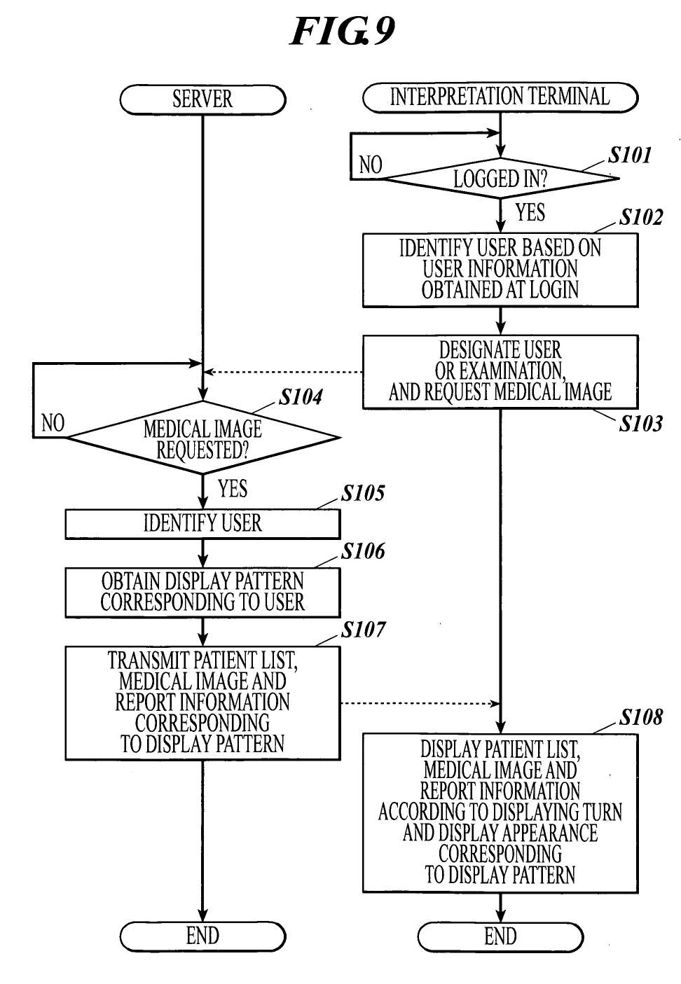

The controlling unit 21 comprises a CPU (Central Processing Unit) and the like. The controlling unit 21 develops a system program stored in the storing unit 26 and a pattern display process program according to the present invention (see FIG. 9) into the RAM 25, and integrally controls processing operations in conjunction with the programs.

The controlling unit 21 stores the medical image generated by the modality 10 in the image DB 30 as an original image. Further, the controlling unit 21 generates various types of images such as a DICOM-compression image (an image compressed according to the DICOM standard), a thumbnail image for index use and the like, and stores them in the image DB 30. At this time, the controlling unit 21 stores information regarding image generation in the image generation information DB 28. Further, the controlling unit 21 controls input/output of the medical images stored in the image DB 30, and delivers the medical image stored in the image DB 30 to an output target, which is the interpretation terminal 40 or the film outputting apparatus 50.

Further, based on the accompanying information added on the medical image, the controlling unit 21 stores the patient information, the examination information, the series information and the image information in the fundamental information DB 27 for updating the fundamental information DB 27. Further, the controlling unit 21 stores a report regarding diagnosis which is generated by each interpretation terminal 40 so as to build a database to be managed.

Further, when the controlling unit 21 receives display pattern information and user information from the interpretation terminal 40, the controlling unit 21 stores them in the display pattern DB 29 so as to relate the display pattern information with the user information. Then, in a pattern display process, the controlling unit 21 obtains display pattern information corresponding to a user assigned in the interpretation terminal 40 from the display pattern DB 29, and obtains patient list information, a medical image and previous report information each of which corresponds to the user from the fundamental information DB 27 and the image DB 30. Then, the controlling unit 21 transmits them to the interpretation terminal 40 via the communicating unit 24.

Further, in regard to display patterns registered in the display pattern DB 29, the controlling unit 21 collects the statistics of frequency of usage of display appearances in the displaying turn, and the controlling unit 21 calculates the most frequently-used display pattern as a standard pattern to be stored in the display pattern DB 29. In other words, it is possible to realize the pattern controlling section by the controlling unit 21.

The inputting unit 22 comprises a keyboard having numeric keys, letter keys, function keys and the like, a mouse and so forth. The inputting unit 22 outputs an operation signal corresponding to an operated key to the controlling unit 21.

The displaying unit 23 is the displaying section comprising an LCD (Liquid Crystal Display) or the like, and it displays various types of screen data such as various types of operation screens, a processing result by the controlling unit 21 and the like.

The communicating unit 24 comprises a communications interface such as a Network Interface Card (hereafter, it is called NIC), a modem or the like, and mutually transmits/receives information to/from an external device on the communication network N. Further, the communicating unit 24 transmits the medical image, which is a storing target, and the report information generated by the interpretation terminal 40, to the image DB 30.

The RAM 25 forms a work area for temporarily storing various types of programs executed by the controlling unit 21 and data regarding these programs.

The storing unit 26 stores the system program, the pattern display process program, data processed by each program and the like.

The fundamental information DB 27 comprises a large-capacity memory. In the large-capacity memory, stored are a patient information table, an examination information table, a series information table and an image information table for storing fundamental information such as the patient information, the examination information the series information and the image information so as to relate each information with each table. Further, the fundamental information DB 27 comprises a report table for storing report information regarding diagnosis. In the report table, for example, stored is various information such as date and time of generating the report, a report file name, an interpretation doctor name, a patient name and the like.

The image generation information DB 28 comprises information tables corresponding to each image type (DICOM-compression image management information table, index image management information table) for storing information regarding generation of the DICOM-compression image generated based on the original image and information regarding generation of the thumbnail image for index use. In each table, for example, stored is information such as date and time at which the image is generated, compression rate and the like. Further, the image generation information DB 28 comprises an image state table for managing generation states of various types of images such as the DICOM-compression image, the thumbnail image for index use. In the image state table, stored is flag data which indicates whether each of the various images has been generated or not.

The display pattern DB 29 is the pattern storing section for storing display pattern information at the time of displaying a medical image, the display pattern information being set corresponding to each user at each interpretation terminal 40, and its report information.

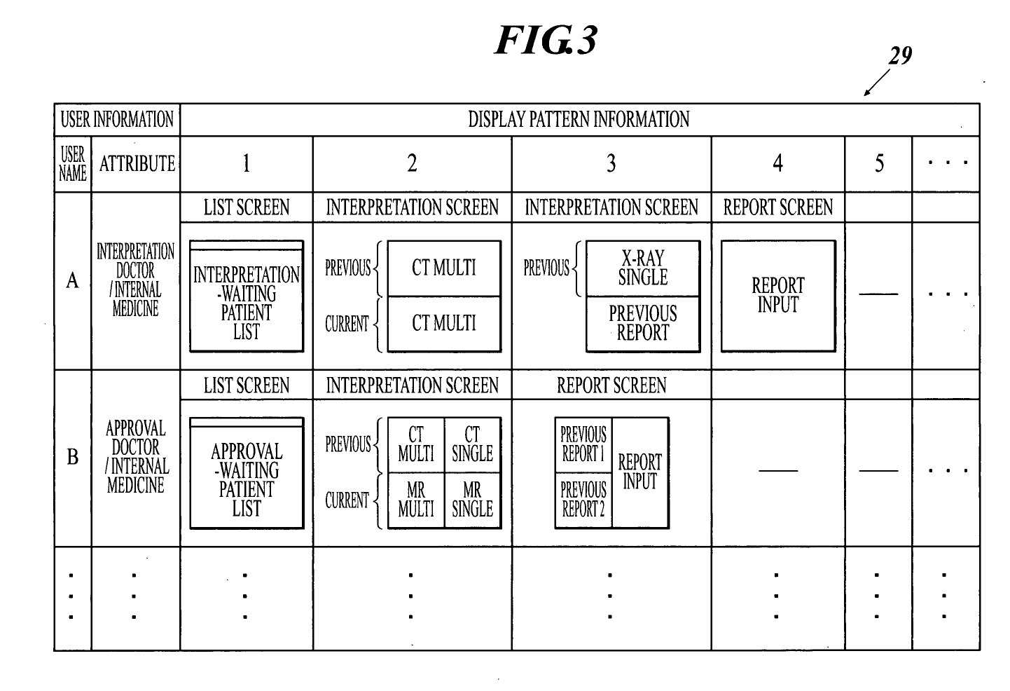

As shown in FIG. 3, in the display pattern DB 29, stored are user information such as a user name (for example, “A”) and a user attribute (for example, “INTERPRETATION DOCTOR/INTERNAL MEDICINE”) and information of a display appearance of a display screen at the time of interpretation, the display appearance being set by the user, so as to relate the information of the display appearance with the user information in the displaying turn. Here, the information of the display appearance includes various types of information, such as a data type to be displayed (patient list information, medical image, report information and the like), an image type of a medical image to be displayed (a type corresponding to an image-generating apparatus such as MR image, CT image, X-ray image or the like; a type corresponding to a generation time such as a previous image, a current image or the like; a type corresponding to the number of displayed images such as a multi image in which a plurality of images are shown in parallel, a single image in which one image is shown, or the like), a display arrangement (for example, an arrangement according to which a screen is divided into two regions so as to show images vertically, an arrangement according to which a screen is divided into two regions so as to show images horizontally, or the like).

For example, in the example shown FIG. 3, in the case of INTERPRETATION DOCTOR A, the display transition is done in the following way: First, a list screen according to a display appearance in which a list of patients to be interpreted is shown on a whole screen is displayed. Second, the screen is divided into two regions vertically so as to provide a multi image form in which a plurality of previous images generated by a CT apparatus are shown in parallel in the upper region, and to provide a multi image form in which a plurality of current images generated by the CT apparatus corresponding to examination at this time are shown in parallel in the lower region. Third, the screen is divided into two regions vertically so as to provide a single image form in which one X-ray image is shown in the upper region, and to show the previous report information corresponding to the X-ray image in the lower region. And fourth, a report screen in which an inputting area for inputting report information corresponding to the interpretation of the current examination is displayed on the whole screen.

The image DB 30 is an NAS (Network Attached Storage) for storing image data of a medical image generated by the modality 10, and stores the original image which is generated by the modality 10, the DICOM-compression image and the thumbnail image which are generated by the server 20 based on the original image, in a folder corresponding to each image type. Further, the image DB 30 stores report information including data of a report document which is created according to the medical image, in a folder for storing a report. In other words, it is possible to realize the information storing section by the image DB 30.

The interpretation terminal 40 is a displaying apparatus for a user such as an interpretation doctor, an approval doctor or the like, to interpret a medical image. The interpretation terminal 40 comprises a displaying section such as an LCD or the like, and displays image data such as a medical image delivered by the server 20, report information and the like.

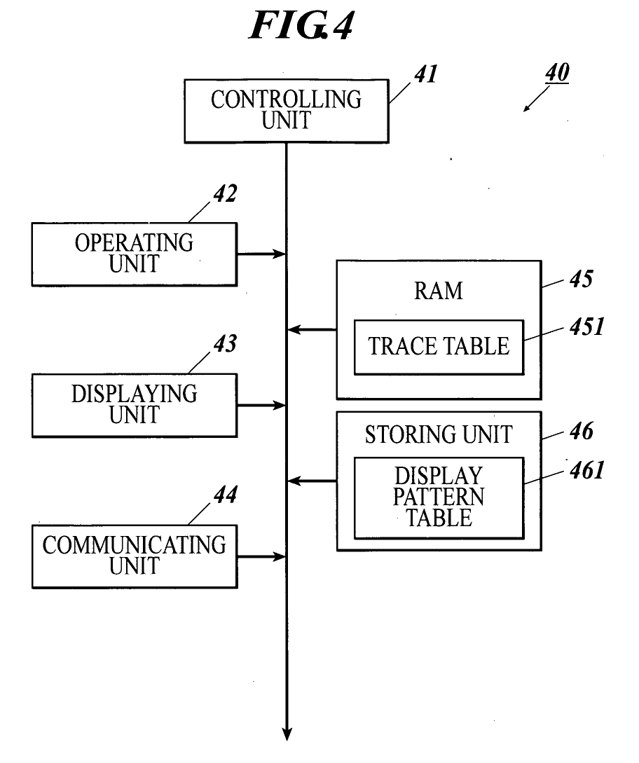

FIG. 4 shows an internal structure of the interpretation terminal 40.

The interpretation terminal 40 comprises a controlling unit 41, an operating unit 42, a displaying unit 43, a communicating unit 44, a RAM 45, and a storing unit 46.

The controlling unit 41 comprises a CPU or the like. The controlling unit 41 develops a system program stored in the storing unit 46, a tracing process program, a pattern display process program and the like into the RAM 45, and integrally controls processing operations in conjunction with these programs.

In the tracing process, a user who is going to perform interpretation is identified based on user information obtained through the operating unit 42 at the time of logging in to the interpretation terminal 40. Then, on each display screen such as a patient list screen, an interpretation screen and a report screen, a display appearance is changed according to a changing instruction inputted by the user through the operating unit 42, and the screen is re-displayed on the displaying unit 43. Further, at each time of changing a display appearance, information of the display appearance is stored in a trace table 451 (which will be described later) of the RAM 45, in the displaying turn. Then, when an instruction to register a display appearance of each display screen and the displaying turn of each display screen is inputted, the display pattern information stored in the trace table 451 and the user information are stored in a display pattern table 461 (which will be described later) of the storing unit 46 so as to relate each other. Each display pattern information stored in the display pattern table 461 is transmitted together with its user information to the server 20 through the communicating unit 44.

In a pattern display process, request information for requesting patient list information, a medical image and previous report information each corresponding to a user identified at the time of login authentication to the interpretation terminal 40 is transmitted to the server 20 through the communicating unit 44, and the medical image and the previous report information are obtained from the server 20. Further, display pattern information corresponding to the identified user is obtained from the display pattern table 461 of the storing unit 46. Then, with the use of the patient list information, the medical images and the report information obtained from the server 20, each display screen such as a patient list screen, an interpretation screen and a report screen is generated based on the obtained display pattern, and each display screen is displayed in the displaying turn which is set by the display pattern, according to the display instruction from the user.

In other words, it is possible to realize the display controlling section by the combination of the tracing process program, the pattern display process program and the controlling unit 41.

The operating unit 42 is the operating section comprising a keyboard, a mouse, a touch panel which is integrated with the displaying unit 43, and the like. The operating unit 42 outputs an operation signal corresponding to an operated key to the controlling unit 41. In other words, it is possible to realize the obtaining section by inputting user information through the operating unit 42.

The displaying unit 43 is the displaying section comprising an LCD or the like. The displaying unit 43 displays patient list information, a medical image, report information and the like delivered from the server 20 according to an assigned display appearance in assigned displaying turn, according to an instruction from the controlling unit 41.

The communicating unit 44 is the communicating section comprising a communications interface such as an NIC, a modem or the like, and mutually transmits/receives information to/from an external device on the communication network N. For example, the communicating unit 44 transmits display pattern information which is to be registered to the server 20, and receives patient information, a medical image, report information and the like from the server 20.

The RAM 45 forms a work area for temporarily storing various types of programs executed by the controlling unit 41 and data related to these programs.

Further, the RAM 45 comprises the trace table 451 for temporarily storing display appearances and displaying turn of the display appearances which are set through the operating unit 42 at the time of displaying a medical image, report information and the like, as a display pattern. A data structure of the trace table 451 is approximately the same as that of the display pattern DB 29 of the server 20 (see FIG. 3) without user information. Therefore, illustration and description of the trace table 451 is omitted here.

The storing unit 46 stores a system program, a tracing process program, a pattern display process program and data processed by each program.

Further, the storing unit 46 comprises the display pattern table 461 for storing display pattern information which is instructed to be registered among the display pattern information temporarily stored in the trace table 451, so as to relate the display pattern information with user information of a user who has instructed the registration thereof. A data structure of the display pattern table 461 is also approximately the same as that of the display pattern DB 29 of the server 20 (see FIG. 3). Therefore, illustration and description of the display pattern table 461 is omitted here.

The film outputting apparatus 50 outputs image data of a medical image delivered from the server 20 on a film.

Next, an operation of the present embodiment will be described.

First, with reference to FIG. 5, a tracing process which is performed by the interpretation terminal 40 will be described. When a medical image, report information and the like are to be displayed, this tracing process is used to trace (to track) display appearances in the displaying turn, the display appearances being set and operated by an interpretation doctor or an approval doctor, and to register it as a display pattern.

In the tracing process shown in FIG. 5, at first, a user such as an interpretation doctor, an approval doctor or the like logs in to the interpretation terminal 40. The interpretation terminal 40 obtains user information (for example, password, user name and the like) which is inputted through the operating unit 42 at the login for user identification, and identifies a user based on the user information (Step S1).

Next, when a user is identified and an instruction to start interpretation is inputted through the operating unit 42, request information for requesting a medical image is generated in the server 20, and the request information is transmitted through the communicating unit 44. When the interpretation terminal 40 receives patient list information from the server 20, a patient list screen is generated by the controlling unit 41 based on the patient list information, and the patient list screen is displayed on the displaying unit 43.

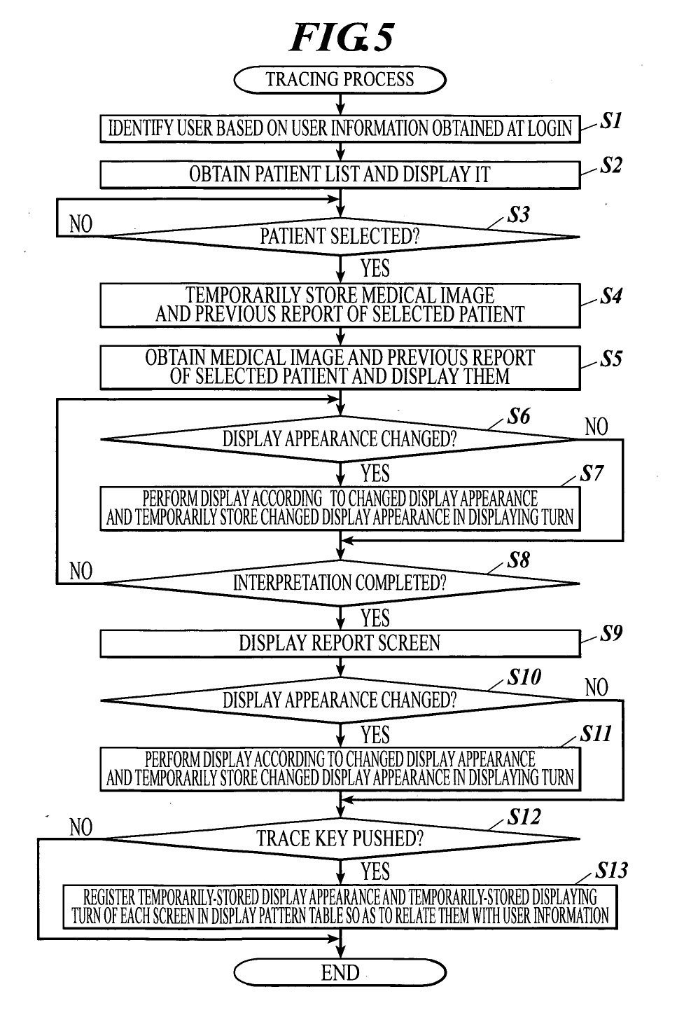

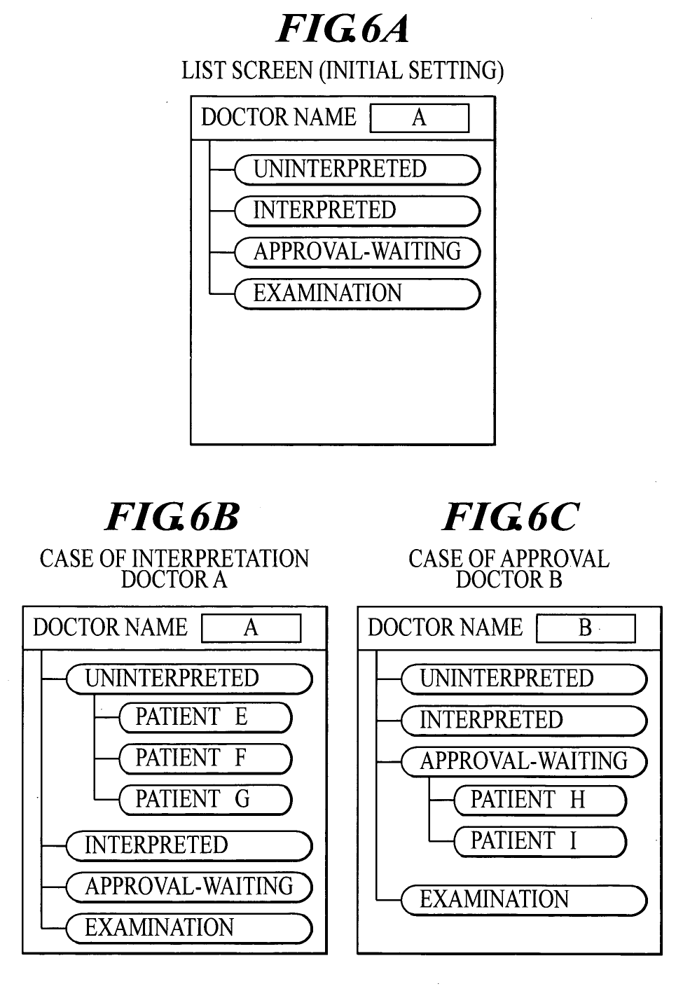

FIGS. 6A to 6C show examples of a patient list screen.

On an initially-set patient list screen, as shown in FIG. 6A, displayed are selection bars corresponding to a usage state of a medical image, such as UNINTERPRETED, INTERPRETED, APPROVAL-WAITING and the like. On the initially-set patient list screen, when an interpretation doctor sees the screen, the selection bar of UNINTERPRETED can be selected, and when an approval doctor sees the screen, the selection bar of APPROVAL-WAITING can be selected. Then, when the selection bar of UNINTERPRETED is selected, as shown in FIG. 6B, patients whose medical images have not yet been interpreted are displayed as a list, or when the selection bar of APPROVAL-WAITING is selected, as shown in FIG. 6C, patients whose medical images have been interpreted by an interpretation doctor and whose report information has been generated are displayed as a list.

In the interpretation terminal 40, a selection of a patient is awaited on the patient list screen, and whether any patient is selected through the operating unit 42 is judged (Step S3). Then, if any patient is selected (Step S3; Y), information of a display appearance on the patient screen is temporarily stored in the trace table 451 (Step S4).

Next, request information for requesting the server 20 a medical image corresponding to the selected patient is transmitted to the server 20. In response to the request from the interpretation terminal 40, in the server 20, a medical image generated at examination of this time (current image), a previous image of the same patient (patient image generated within a predetermined period) and report information of the previous image (hereafter, it is called previous report information) are read out from the image DB 30 as medical images of the patient corresponding to the request, and transmitted to the interpretation terminal 40.

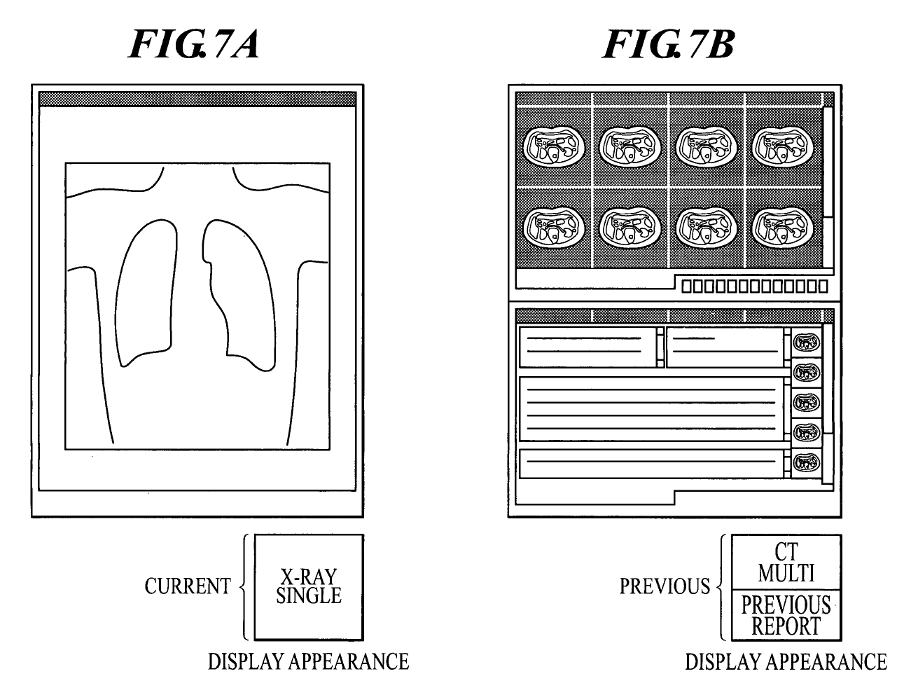

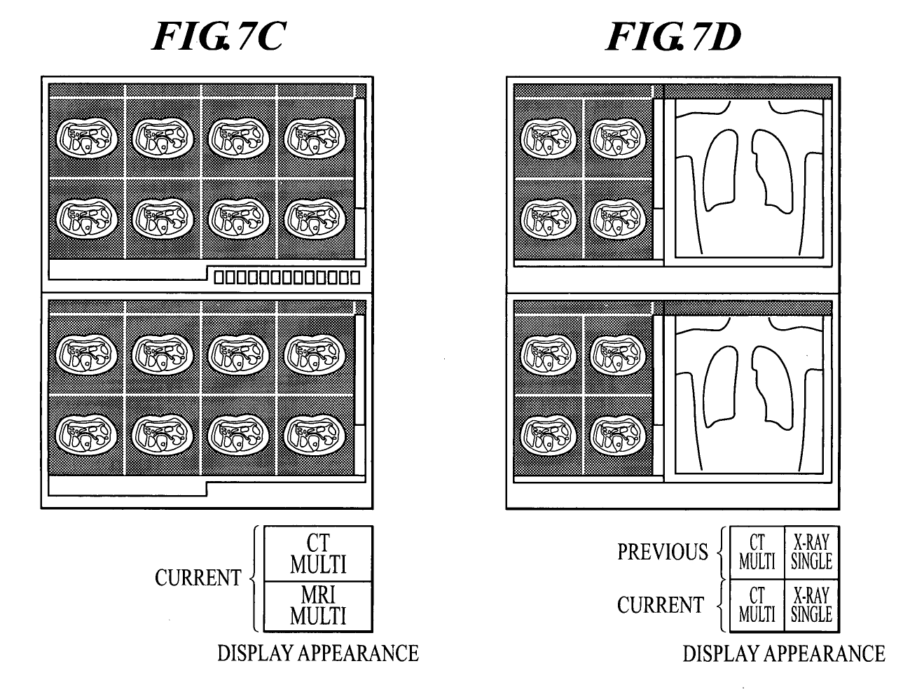

In the interpretation terminal 40, when the current medical image, the previous medical image, and the previous report information are obtained from the server 20, the obtained medical images are displayed on the interpretation screen according to a display appearance of the initial setting (Step S5). For example, if the current image is displayed in a single image form according to the display appearance of the initial setting, an interpretation screen shown in FIG. 7A is displayed. On this interpretation screen, according to user's region to be diagnosed, an image type to be used for the diagnosis and the like, it is possible to do the operation to change a display appearance through the operating unit 42 so as to make it easier to interpret. With the changing operation, it is possible to select a data type and an image type of a medical image to be displayed, such as a previous image of the same patient, previous report information, a CT image, an X-ray image and the like. Further, it is possible to select a display form such as a single display form in which only one piece of data is displayed on one screen, a multi display form in which a plurality of images and a plurality of pieces of report information are displayed in parallel, and the like.

For example, by selecting a display menu while the interpretation screen shown in FIG. 7A is being displayed, as shown in FIG. 7B, it is possible to provide a display appearance in which a screen is divided into two regions vertically so as to provide the multi display form in the upper region and to display previous report information in the lower region. Similarly, as shown in FIG. 7C, it is possible to show a display appearance in which the multi display form of MRI images is provided in the upper region and the multi display form of CT images is provided in the lower region, and, as shown in FIG. 7D, it is possible to show a display appearance in which the vertically-divided screens are further divided horizontally into four regions in total, so as to provide the multi display form of CT images in the left regions and to provide the single display form of X-ray images in the right regions, where the upper regions show previous images and the lower regions show current images.

Next, whether a change of a display appearance is instructed on the interpretation screen is judged (Step S6). If a change of a display appearance is instructed through the operating unit 42 (Step S6; Y), a display appearance is changed according to the instruction and the interpretation screen is re-displayed according to the changed display appearance. Further, information of the changed display appearances is temporarily stored in the trace table 451 so as to relate the display appearances with its displaying turn (Step S7) and the operation proceeds to a process of Step S8.

On the other hand, if a change of a display appearance is not instructed (Step S6; N), the operation proceeds to a process of Step S8, in which whether an instruction to complete the interpretation and to display a report screen is inputted is judged (Step S8). If an instruction to complete the interpretation is not inputted (Step S8; N), the operation goes back to the process of Step S6, and if an instruction to complete the interpretation is inputted (Step S8; Y), a report screen for inputting report information with respect to the examination of this time is displayed on the displaying unit 43 according to a display appearance of the initial setting (Step S9).

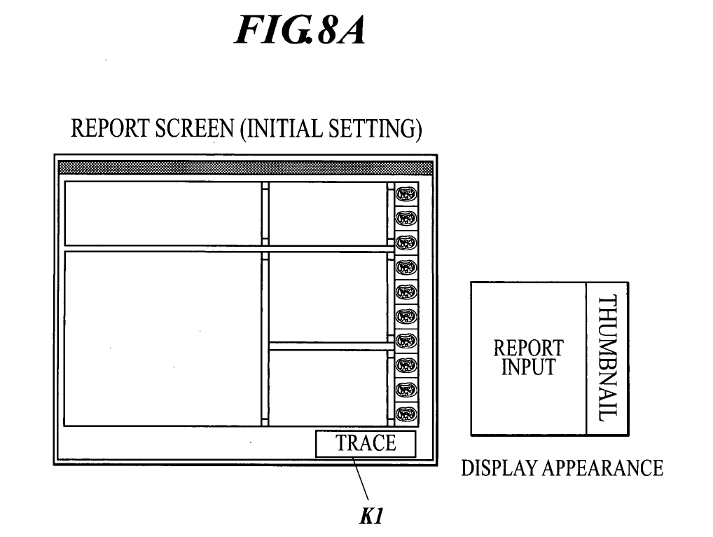

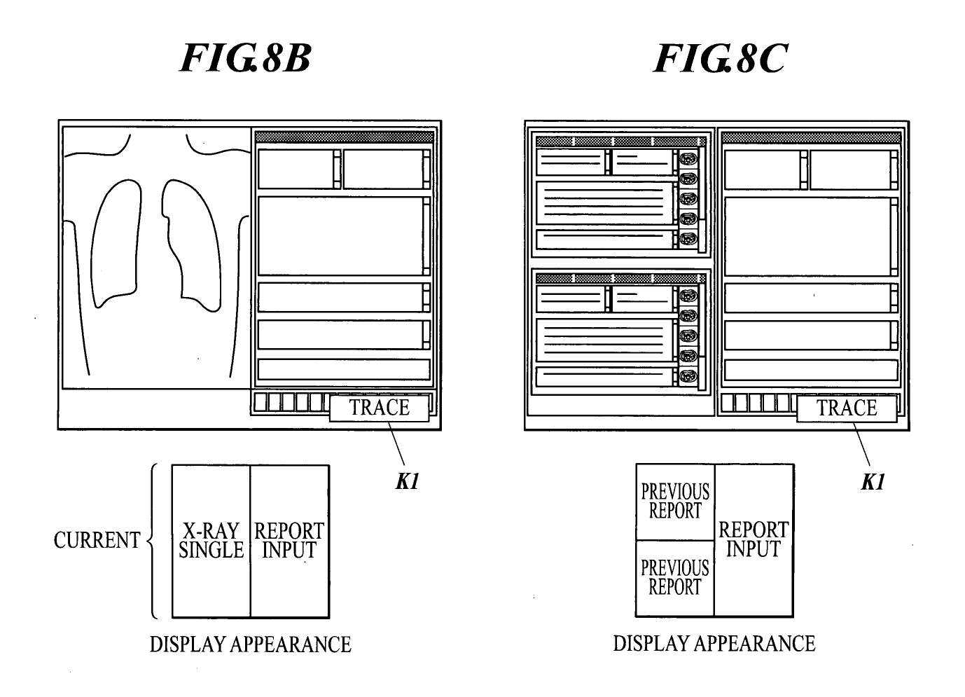

FIGS. 8A to 8C show examples of the report screen.

For example, if a display appearance of the initial setting is to display an inputting area on the whole screen, the inputting area being used for inputting report information with respect to the examination of this time, a report screen shown in FIG. 8A is displayed. On this report screen, it is possible to do the operation from the display menu to change a display appearance so as to make it easier to generate a report. With the changing operation, it is possible to select a display of previous report information for reference use, a display of a current image and a thumbnail image together with current report information, and the like.

For example, by selecting a display menu while the report screen shown in FIG. 8A is being displayed, as shown in FIG. 8B, it is possible to divide a screen into two regions horizontally so as to perform the single display form of an X-ray image in the left region and to display an area for inputting report in the right region. Further, as shown in FIG. 8C, with the left region further divided into two regions vertically, it is possible to display previous report information in the left regions so that date of previous report information displayed in the upper region and date of previous report information displayed in the lower region are different from each other.

Further, a trace key k1 is provided at the bottom of the report screen. By pushing the trace key k1, it is possible to do the registration of a display appearance of each display screen and its displaying turn from the patient list screen through the interpretation screen to the report screen with respect to a user. After a user inputs report information on the report screen, if he/she wishes to register the display appearances and the displaying turn of the display screens until that moment, he/she pushes the trace key k1. Here, it is possible to substitute a report registration key which is pushed at the time of registering a report, for this trace key k1.

Next, whether a change of a display appearance is instructed on the report screen is judged (Step S10). If a change of a display appearance is instructed through the operating unit 42 (Step S10; Y), a display appearance is changed according to the instruction, and the report screen is re-displayed according to the changed display appearance. Further, information of the changed display appearances is temporarily stored in the trace table 451 in its displaying turn (Step S11) and the operation proceeds to a process of Step S12.

On the other hand, if a change of a display appearance is not instructed (Step S10; N), whether the trace key k1 is pushed is judged in Step S12. If the trace key k1 is not pushed (Step S12; N), the present process is completed, and if the trace key k1 is pushed (Step S12; Y), the display appearances and the displaying turn of each display screen which are temporarily stored in the trace table 451 are stored in the display pattern table 461 as a display pattern corresponding to a user so as to relate the display pattern and user information of the user identified in Step S1 (Step S13). Then, when the registration of a display pattern is completed, the present process is completed.

The display pattern information registered in the display pattern table 461 is thereafter transmitted to the server 20, and stored in the display pattern DB 29.

In the server 20, at each time that a certain period has passed, the statistics of display appearances regarding the display pattern information stored in the display pattern DB 29 is collected in the displaying turn, and a display pattern having the highest frequency of use is calculated. A unit of the display pattern regarding which statistics is to be collected can be suitably set according to a purpose. For example, the unit can be an interpretation doctor, an approval doctor, a diagnosis department to which doctors belong, an image type to be used for interpretation, and the like. Then, when a display pattern having the highest frequency of use is calculated according to the statistics, the display pattern is registered to the display pattern DB 29 as a standard pattern corresponding to the unit based on which the statistics is collected.

By using this standard pattern, it is possible to standardize the procedure of interpretation and approval. For example, by using an interpretation doctor of internal medicine as a statistics unit, a display pattern having the highest frequency of use is calculated as a standard pattern. Then, by displaying display screens at the time of interpretation according the display appearance corresponding to the standard pattern in the displaying turn of the standard pattern, it is possible to unify the procedure of interpretation at the internal medicine.

Next, with reference to FIG. 9, a pattern display process for displaying a medical image, report information and the like according to a registered display pattern after the display pattern is registered will be described. FIG. 9 is a flowchart illustrating the pattern display process performed by the server 20 and the interpretation terminal 40.

In the pattern display process shown in FIG. 9, first, when a user logs in to the interpretation terminal 40 (Step S101; Y), the user is identified with a user identification performed at the login (Step S102). Next, when an instruction to start the interpretation is inputted through the operating unit 42, the identified user (or examination) is designated and requirement information for requiring a medical image is transmitted to the server 20 (Step S103).

In the server 20, whether a medical image is requested by the interpretation terminal 40 is judged (Step S104). If the request information of a medical image is received from the interpretation terminal 40 (Step S104; Y), the user designated by the interpretation terminal 40 is identified (Step S105).

When the user is identified, in the server 20, display pattern information corresponding to the identified user is obtained from the display pattern DB 29 (Step S106). Next, the server 20 transmits patient list information corresponding to a patient to be interpreted to the interpretation terminal 40. Further, according to a control by the server 20, based on the obtained display pattern information, a medical image of an image type which is necessary for the display according to the display pattern (including a previous image and a current image), and previous report information are transmitted from the image DB 30 to the interpretation terminal 40 (Step S107).

Meanwhile, in the interpretation terminal 40, display pattern information corresponding to the user identified in Step S102 is obtained from the display pattern table 461. Then, when a medical image and previous report information are received from the server 20, by using the received medical image and previous report information, a patient list screen, an interpretation screen and a report screen are generated according to display appearances corresponding to the obtained display pattern. Then, according to a display instruction inputted through the operating unit 42, the generated patient list screen, the generated interpretation screen and the generated report screen are displayed in the displaying turn corresponding to the display pattern (Step S108). Then, the present process is completed.

As above, in the present embodiment, since it is possible to set a display appearance and displaying turn of each display screen during interpretation as a display pattern with respect to each user, it is possible to. customize a display pattern according to a diagnosis purpose of each user and personal preference. Therefore, it is possible to display a medical image and report information according to a display appearance in the displaying turn so as to make the interpretation easy, and thereby, it is possible to make the interpretation operation more efficient.

Further, by patterning the display, even an interpretation doctor having little experience can do the interpretation efficiently by performing the interpretation according to a display pattern which is set by an experienced interpretation doctor or an experienced approval doctor. Thereby, it is possible to improve the whole operation efficiency and a diagnosis level.

Further, in the server 20, since statistics of a display pattern registered by each interpretation terminal 40 is collected and a display pattern having the highest frequency of use is registered as a standard pattern, it is possible to standardize a display pattern. Thereby, it is possible to unify the operation procedure such as interpretation, approval and the like according to a diagnosis department unit, a hospital unit and the like.

Here, the described content in the present embodiment is a suitable example of the medical image management system 100 to which the present invention is applied, and the present invention is not limited to the content.

In the above, described is the case that the pattern controlling section and the pattern storing section are realized by the server 20, the information storing section is realized by the image DB 30, and the obtaining section and the display controlling section are realized by the interpretation terminal 40. However, each section may be realized by any device.

And so forth, the detailed structure and operation of the medical image management system 100 in the present embodiment may be suitably changed without departing the gist of the present invention.

The entire disclosure of a Japanese Patent Application No. Tokugan 2003-385318 filed on Nov. 14, 2003, including specifications, claims, drawings and summaries are incorporated herein by reference in their entirety.

1. A medical image management system comprising:
an information storing section for storing a medical image in which an image of a subject is generated and report information regarding diagnosis;
a displaying section for displaying at least one of the medical image and the report information each of which is obtained from the information storing section;
an operating section for setting an image format of a display screen and displaying turn of the display screen which are used to display at least one of the medical image and the report information on the displaying section;
a pattern storing section for storing the display appearance of the display screen and the displaying turn of the display screen which is set through the operating section, so as to relate the display appearance and the displaying turn with user information of a first user who has operated the operating section;
an obtaining section for obtaining user information of a second user who performs an interpretation; and
a display controlling section for obtaining at least one of the medical image and the report information from the information storing section during the interpretation, for obtaining display pattern information corresponding to the user information obtained by the obtaining section from the pattern storing section, and for displaying at least one of the medical image and the report information on the displaying section according to the display appearance and the displaying turn corresponding to the obtained display pattern information.

2. The system of claim 1, further comprising a pattern controlling section for collecting statistics of the display pattern information stored in the display pattern storing section, for calculating a display pattern having highest frequency of use as a standard pattern, and for storing information of the standard pattern in the display pattern storing section.
